(19) United States
(12) Patent Application Publication
(10) Pub. No.: US 2009/0004660 A1
(43) Pub. Date: Jan. 1, 2009

(54) METHOD FOR DETECTING NEUROBLASTOMA AND ITS MALIGNANCY AND METHOD FOR SUPPRESSING THE SAME

(76) Inventors: Johji Inazawa, Tokyo (JP); Issei Imoto, Tokyo (JP); Yurlko Sugino, Tokyo (JP)

Correspondence Address: BIRCH STEWART KOLASCH & BIRCH, PO BOX 747, FALLS CHURCH, VA, 22040-0747, US

(21) Appl. No.: 12/126,637

(22) Filed: May 23, 2008

(30) Foreign Application Priority Data
Jun. 28, 2007 (JP) 2007-169875

(51) Int. Cl. C12Q 1/68; C40B 30/04; C12N 5/06; C07H 21/04; C07K 14/00

(52) U.S. Cl. 435/6; 506/9; 435/375; 536/23.5; 530/350; 536/24.5

(57) ABSTRACT

An object of the present invention is to provide a method for detecting cancer and a cell growth inhibitor through identification of genes exhibiting characteristic behavior in the cases of cancer such as neuroblastoma. The present invention provides a method for detecting cancer, which comprises detecting canceration including malignancy of a specimen through detection of inactivation of a gene in the q22 region of chromosome 14 in the specimen.

### TECHNICAL FIELD

The present invention relates to a method for detecting cancer by detecting gene alterations that exist in specific chromosomal regions for the purpose of early diagnosis of neuroblastoma through observation of the genotype thereof.

### BACKGROUND ART

Neuroblastoma is a tumor that is generated in adrenal glands and sympathetic ganglia. The onset mostly takes place in children aged 5 years or younger; however, the onset can also take place after 5 years of age on rare occasions, and it is also found in newborns. Since adrenal glands and sympathetic ganglia are developed from a common tissue, called the neural crest, in the fetal period, cells derived from the neural crest are considered to be the origin of neuroblastoma (Brodeur., et al., Nat Rev Cancer, 3, 203-216, 2003; and Westermann et al., Cancer Lett, 184, 127-147, 2002).

Generally, neuroblastoma occurring in children over 1 year is often advanced and requires intensive treatment which is a combination of surgical operation, chemotherapy, and radiotherapy. In particular, in cases of stage 4 or in cases where MYCN oncogene is amplified, aggressive treatments using hematopoietic stem cell transplantation (such as bone marrow transplantation and peripheral blood stem cell transplantation) have been carried out. However, in cases of stage 4 neuroblastoma, the 5-year survival rate is only about 30% even with such aggressive treatments in combination with hematopoietic stem cell transplantation. Accordingly, it has been desired to discover a causative gene of the occurrence of neuroblastoma and to elucidate the functions for development of a new effective therapeutic method based on the understandings.

### DISCLOSURE OF THE INVENTION

Successful elucidation of the mechanism of canceration of neural-crest-derived cells at the gene level will enable detection of canceration of neural-crest-derived cells at the gene level, diagnosis of the malignancy of neuroblastoma, and suppression of the advancement thereof. Furthermore, it will also enable establishment of methods for selecting and developing drugs, as well as therapeutic methods based on such mechanisms. Specifically, this object can be achieved by identifying genes exhibiting characteristic behavior observed in neuroblastoma cases and then carrying out technical examination mainly targeting such genes. Hence, an object to be achieved by the present invention is to provide a method for detecting cancer and a cell growth inhibitor through identification of genes exhibiting characteristic behavior in the cases of cancer such as neuroblastoma.

Comparative Genomic Hybridization (CGH), or associated Bacterial Artificial Chromosome array-based Methylated CpG island Amplification (BAMCA), is the best method for conveniently and rapidly analyzing genetic abnormalities accompanying amplification or deletion of numerous genes in the genome or inactivation of genes. To analyze genetic abnormalities in the genome involved in canceration and higher cancer malignancy, the present inventors have selected 4500 types of BAC/PAC DNA to be subjected to CGH assay (MCG Whole Genome-4500; Inazawa J., et al., Cancer Sci. 95, 559-563, 2004). As a result, the present inventors have succeeded in identification of a cancer-associated gene that promotes canceration of neural crest cells; that is, a Prostaglandin E receptor 2 (PTGER2) gene. Moreover, the present inventors have succeeded in discovering that inactivation of the PTGER2 gene, and specifically a decrease in the PTGER2 protein, significantly promotes the proliferation of neuroblastoma and that the increased level of a transcript or the protein of the PTGER2 gene results in significantly decreased levels of neuroblastoma proliferation. Thus, the present inventors have completed the present invention.

The present invention provides a method for detecting cancer, which comprises detecting canceration including malignancy of a specimen through detection of inactivation of a gene in the q22 region of chromosome 14 in the specimen.

Preferably, the gene is a PTGDR gene or a PTGER2 gene.

Preferably, amplification of an MYCN gene is further detected in the specimen.

Preferably, the gene inactivation is inactivation due to methylation of a CpG island.

Preferably, the gene inactivation is inactivation due to acetylation status of a histone H4 protein or a histone H3 protein, or trimethylation status of histone H3K9.

Preferably, the specimen is a cell derived from the neural crest.

Preferably, cancer is neuroblastoma.

Preferably, the gene inactivation is detected by using a DNA chip method, a Southern blot method, a Northern blot method, a real-time RT-PCR method, a FISH method, a CGH method, an array CGH method, a bisulfite sequence method, or a COBRA method.

The present invention further provides a method for inhibiting cell growth, which comprises introducing a PTGDR gene, a PTGER2 gene or a protein which is an expression product of said gene into cells in vitro.

The present invention further provides a cell growth inhibitor, which comprises a PTGDR gene, a PTGER2 gene, or a protein which is an expression product of said gene.

The present invention further provides a method for activating cell growth, which comprises introducing an siRNA, an shRNA, an antisense oligonucleotide, or a loss-of-function type gene of a PTGDR gene or a PTGER2 gene into tumor cells in vitro.

The present invention further provides a cell growth activating agent, which comprises an siRNA, an shRNA, an antisense oligonucleotide, or a loss-of-function type gene of a PTGDR gene or a PTGER2 gene.

The present invention further provides a method for inhibiting cell growth, which comprises in vitro accumulation of cAMP in a specimen.

The present invention further provides a screening method of a substance, which comprises: contacting a test substance with a neuroblastoma cell showing lowered expression of a PTGDR gene or a PTGER2 gene due to methylation of a CpG island of the PTGDR gene or the PTGER2 gene; detecting expression of the PTGDR gene or the PTGER2 gene; and, if the gene expression thereof is higher than that of a system without the contact of the test substance, selecting the test substance as an antitumor substance capable of activating the PTGDR gene or the PTGER2 gene through demethylation of the CpG island of the PTGDR gene or the PTGER2 gene.

The present invention further provides a screening method of a substance, which comprises: contacting a test substance with a neuroblastoma cell showing lowered expression of a PTGDR gene or a PTGER2 gene due to hypoacetylation of a histone H3 or H4 protein or methylation of a lysine residue at position 6 (K9) of a histone H3 protein; detecting expression of the PTGDR gene or the PTGER2 gene; and, if the gene expression thereof is higher than that of a system without the contact of the test substance, selecting the test substance as an antitumor substance capable of activating the PTGDR gene or the PTGER2 gene through enhancement of acetylation of the histone H4 protein.

### BRIEF DESCRIPTION OF THE DRAWINGS

FIG. 1 shows the genomic structure of the PTGDR gene and the PTGER2 gene, and expression of these genes in neuroblastoma cell lines.

A: Genomic structure of the PTGDR gene and the PTGER2 gene. Open and filled boxes represent untranslated regions and coding exonic sequences respectively. Hatched boxes indicate 1479 bp and 1482 bp CpG islands, respectively.

B: RT-PCR analysis of the PTGDR gene and the PTGER2 gene in normal brain, adrenal gland, and neuroblastoma cell lines with (+) or without (−) amplification of the MYCN gene. Expression of GAPDH is shown as a control.

C: Results of RT-PCR analysis of the PTGDR gene and the PTGER2 gene in neuroblastoma cell lines with (+) and without (−) treatment with 5-aza-dC and/or TSA. Expression of GAPDH is shown as a control. Expressions of both genes were restored by treatment with 5-aza-dCyd in three types of cell lines. Expression of the PTGER2 gene was restored by treatment with TSA alone in three types of cell lines.

FIG. 2 shows association of DNA methylation and histone modification with expression status of the PTGER2 gene.

A: Map of the CpG islands (stippled bars) around the exon 1 of the PTGDR and PTGER2 genes. CpG sites are represented by vertical marks. Fragments of each gene (Regions 1-3) were examined for methylation by COBRA and bisulfite sequencing (indicated by closed arrows). Fragments of the PTGRE2 gene examined in promoter assays (Regions 1-4) are indicated by horizontal bars. The region analyzed by ChIP-PCR is indicated by arrowheads.

B: Top: Results of bisulfite sequencing in PTGDR gene- and PTGER2 gene-nonexpressing neuroblastoma cell lines (IMR32, GOTO, and SJ-N-CG) and expressing neuroblastoma cell lines (KP-N-SILA or SH-SY5Y), and four types of primary neuroblastoma. Open and filled squares represent unmethylated and methylated CpG sites, and each row represents those from a single clone. TaqI restriction sites are indicated by black arrowheads. Black (MSP) and white (USP) arrows indicate the positions of primer sequences designed to amplify methylated and unmethylated alleles. Bottom: Results of COBRA experiments involving Region 3 of the CpG island of PTGDR and Region 3 of the CpG island of PTGER2 in neuroblastoma cell lines with (+) or without (−) expression of each gene. PCR products were digested by TaqI. Arrows indicate unmethylated alleles. Arrowheads indicate methylated alleles.

C: Promoter activity of the PTGER2 CpG island. pGL3 empty vectors (mock) and reporter containing one of different sequences within CpG island Regions 1-4, were constructed, and were transfected into PTGER2 gene-expressing cells (SH-SY5Y) and PTGER2 gene-nonexpressing cells (SJ-N-CG). Luciferase activities were normalized versus a control. The data presented are the means± s.d. of three independent experiments.

D: ChIP assay showing the status of histone acetylation and methylation of the PTGER2 promoter region in neuroblastoma cells. Left: Immunoprecipitation method of DNA-protein complex using antibodies against acetylated histone H3 (Ac-H3), acetylated histone H4 (Ac-H4), and trimethylated histone H3-lysine 9 (3Me-H3K9). Experiments were performed using crosslinked extracts from PTGER2 gene-expressing cell lines (SH-SY5Y) and from PTGER2 gene-nonexpressing cell lines (SJ-N-CG and GOTO). Immunoprecipitated samples containing the PTGER2 promoter region were amplified by PCR. A portion of the sonicated chromatin before immunoprecipitation (input) was provided as a positive control for normalization. An immunoprecipitated product without antibody was provided as a negative control. Right: Quantitative analysis of ChIP-PCR products. Bands produced from ChIP-PCR products were quantified with a densitometry (LAS-3000, FUJIFILM Corporation). DNAs binding to acetylated histone H3 and H4 within the PTGER2 promoter were decreased in SJ-N-CG and GOTO cell lines. Trimethylated histone H3K9 was increased as compared with SY5Y. This suggests association of methylation pattern within the CpG island and histone modification within the promoter region with regulation of the expression of the PTGER2 gene in neuroblastoma cells.

FIG. 3 shows methylation and expression status of PTGER2 in primary neuroblastoma.

A: Results of MSP experiments for primary samples of neuroblastoma. Primer sets for methylated and unmethylated alleles were designed within Region 3 of the CpG island of PTGER2 shown in FIG. 2B, and amplification was preformed. Arrowheads indicate methylated alleles of two types of tumors.

B: PTGER2 methylation status of primary neuroblastoma, compared with tumor stage (left) and MYCN amplification status (right). Methylation status was determined by MSP. Left: The PTGER2 CpG island was methylated in four of 37 cases of stage 1, 2, 3 or 4S tumors (10.8%), while 8 of 12 cases (66.7%) of stage 4 tumors showed methylation (p=0.0004, Fisher's exact test). Right: Methylation of the PTGER2 CpG island was found in eight of nine cases (88.9%) with amplification of the MYCN gene, while only four of 40 cases (10%) without amplification of the MYCN gene showed methylation of the PTGER2 CpG island (P<0.0001, Fisher's exact test).

FIG. 4 shows effects of expression of the PTGER2 gene on proliferation of neuroblastoma. Colony-formation assays using neuroblastoma cell lines which lack PTGER2 gene expression (GOTO and SJ-N-CG). The cells were transiently transfected with a Myc-tagged vector containing PTGER2 (pcDNA3.1-PTGER2-Myc), or an empty vector (pcDNA3.1-empty), and were subjected to drug selection for 2 to 3 weeks under the presence of G418. Left: Two (SJ-N-CG) or three (GOTO) weeks after transfection and subsequent formation of drug-resistant colonies, the colonies formed by PTGER2 gene-transfected cells were less numerous than those formed by empty vector-transfected cells. Right: Quantitative analysis of colony formation. Colonies larger than 2 mm were counted, and results are presented as means± s.d. of three separate experiments. Statistical analysis used the Mann-Whitney U-test (:a, P<0.05 versus empty vector-transfected cells).

FIG. 5 shows effects of a cell-permeable cAMP analog, 8-Br-cAMP, on proliferation of neuroblastoma. Wild-type GOTO and SJ-N-CG cells, and SJ-N-KP and KP-N-SIFA cells, which lack expression of PTGER2, were cultured for 72 hours with various concentrations of 8-Br-cAMP, and cell viability was determined by WST assay. Statistical analysis used Scheffe's tests which is one type of variance analysis (:a, p<0.05 versus cells treated with vehicle).

### BEST MODE FOR CARRYING OUT THE INVENTION

Hereafter, the present invention will be described in detail.

### (1) Method for Detecting Cancer

The method for detecting cancer according to the present invention comprises detecting canceration including the malignancy of a specimen, through detection of inactivation of a gene in the q22 region of chromosome 14. Preferably, the gene to be detected is a PTGDR gene or a PTGER2 gene.

As a result of the human genome project, a transcript of the human PTGER2 gene is already known. The human PTGER2 gene is located in chromosome 14q22.1 (Duncan A M., et al., Genomics, 25, 740-742, 1995). The protein encoded by the human PTGER2 gene is a receptor of prostaglandin E2 (PGE2) and functions to mediate the PGE2 signaling (Narumiya S., et al., Physiol Rev, 79, 1193-1226, 1999). However, the fact that the human PTGER2 gene is an important cancer-associated gene involved in the onset or malignancy in human neuroblastoma has not yet been known.

As described above, the present detection method is a method which comprises detecting inactivation of the PTGER2 gene in neural crest cells and neuroblastoma cells.

Appropriate neural crest cells or neuroblastoma cells for the detection of the inactivation of the PTGER2 gene are tissue cells biopsied from specimen donors.

The tissue cells to be used as specimens may be either neural crest cells from a healthy subject or a cancer tissue cells from a neuroblastoma patient. However, in reality, possible subjects are mainly cells derived from: pathologic tissue of adrenal glands and sympathetic ganglia having a lesion suspected to be malignant as confirmed by a test or the like; tissue that is confirmed to be neuroblastoma and for which determination of malignancy or the stage progression thereof is required; or the like.

When the inactivation of the PTGER2 gene is confirmed in the “pathologic tissue of adrenal glands and sympathetic ganglia having a lesion suspected to be malignant as confirmed by a test or the like”, it is revealed that the pathologic tissue is undergoing a process toward canceration or is already in the malignant state, and that the malignancy thereof is increasing. Thus, the need to carry out immediate full-scale treatment (such as lesion removal by operation or the like and full-scale chemotherapy) is demonstrated. Moreover, when the inactivation of the PTGER2 gene is confirmed in the “tissue that is confirmed to be neuroblastoma and for which determination of malignancy or the stage progression thereof is required”, it is revealed that the malignancy of the cancer tissue is increasing. Hence, the need to carry out immediate full-scale treatment (such as lesion removal by operation or the like or full-scale chemotherapy) is demonstrated. A neuroblastoma tissue sampled as a specimen can be subjected to the present detection method after applying necessary treatment such as with the preparation of DNA or RNA from the sampled tissue.

The causative factors of lowered expression level of the PTGER2 gene are largely divided into two factors: a) inactivation due to methylation of CpG island of the PTGER2 gene; and b) inactivation due to hypoacetylation of the histone H4 protein. Hereunder, these two factors will be briefly described.

### (a) Inactivation Due to Methylation of CpG Island of PTGER2 Gene

It has been reported that dense methylation of CpG-rich promoters and exon regions causes transcriptional inactivation (Bird, A P et al., Cell, 99, 451-454, 1999). In cancer cells, CpG islands are densely methylated more frequently as compared to other regions. Hypermethylation of promoter regions are deeply associated with inactivation of cancer suppressor genes in cancer (Ehrlich, M., et al., Oncogene, 21, 6694-6702, 2002).

As described later, actually, the CpG island existing in the exon of the PTGER2 gene has a promoter activity; however, it was revealed that in vitro methylation of this region causes loss of the promoter activity. Moreover, the methylation status of the CpG island was strongly correlated with lowered expression of the PTGER2 gene in some neuroblastoma cells. Further, the CpG island was successfully demethylated by culturing neuroblastoma cells under the presence of a demethylating agent, 5-azadeoxycytidine (5-aza-dC). As a result, the expression of the PTGER2 gene was successfully restored. These results revealed that highly frequent methylation (hypermethylation) of the CpG island is one of the causative factors of highly frequent suppression of cancer suppressor genes in neuroblastoma.

### (b) Inactivation Due to Hypoacetylation of Histone H4 Protein

It is known that modification of histone proteins is associated with suppression of gene expression induced by DNA methylation (Cameron et al., Nucleic Acids Res., 2001, 29, 4598-4606).

Trichostatin A (TSA) is a histone deacetylase inhibitor, which is an important tool for analyzing the relation between acetylation and gene expression. When cultured under the presence of TSA, some neuroblastoma cells showed increased level of PTGER2 gene expression (described later). Accordingly, it can be concluded that the acetylation status of the histone H3 or H4 protein which binds to DNA within a cell is increased under the presence of TSA. Further, it was also revealed that activation of the PTGER2 gene expression induced by acetylation of the histone H3 or H4 protein are independent of methylation of CpG islands in neuroblastoma.

(c) Regarding specimen cells showing lowered expression level of the PTGER2 gene, such classification into two types of causative factors (a) and (b) of inactivation by the abovementioned method is very useful for carrying out the most appropriate treatment (selection of antitumor agent(s) to be administered) for specimen donors. Specifically, the type of above causative factors can be classified through the abovementioned detection method, in which specimen cells showing lowered expression level of the PTGER2 gene (cancer tissue-derived primary cancer cells) are treated with a demethylating agent (such as 5-azadeoxycytidine) or an acetylation enhancer (such as Trichostatin A) and are examined for the restoration of the gene expression level.

That is to say, if the PTGER2 gene expression level is restored by treating specimen cells with a demethylating agent, the causative factor of the gene suppression in the specimen cells is methylation of CpG island, in which case a reasonable antitumor effect can be expected by administering the specimen donor with a drug having a demethylating function. Moreover, if the PTGER2 gene expression level is restored by treating specimen cells with an acetylation enhancer, the causative factor of the gene suppression in the specimen cells is hypoacetylation in the histone H3 or H4 protein, in which case a reasonable antitumor effect can be expected by administering the specimen donor with a drug having an acetylation enhancing function. Furthermore, if these two reactions are both observed, the causative factor of the suppression of the PTGER2 gene expression in the specimen cells is concluded to be both methylation and hypoacetylation mentioned above, in which case a reasonable antitumor effect can be expected by administering both of such demethylating agent and acetylation enhancer.

### (2) Method for Inhibiting Cell Growth and Cell Growth Inhibitor

According to the present invention, there are further provided a method for inhibiting cell growth which comprises introducing a PTGDR gene, a PTGER2 gene, or a protein which is an expression product of such a gene into cells in vitro, and a cell growth inhibitor comprising said gene or protein.

For handling the PTGDR gene or the PTGER2 gene, cDNAs obtained from cultured cells through publicly known methods to those skilled in the art may be used, or enzymatically synthesized ones through PCR method may be also used. When DNA is obtained through PCR method, PCR is performed using human chromosomal DNA or cDNA library as a template, and primers designed to amplify a nucleotide sequence of interest. DNA fragments amplified through PCR can be cloned in an appropriate vector which can proliferate in a host such as E. coli.

Manipulations such as preparation of detection probes or primers for the PTGDR gene or the PTGER2 gene and cloning of target genes are already known to those skilled in the art. For example, such manipulations can be performed according to methods described in Molecular Cloning: A Laboratory Mannual, 2^{nd} Ed., Cold Spring Harbor Laboratory, Cold Spring Harbor, N.Y., 1989, Current Protocols in Molecular Biology, Supplement 1 to 38, John Wiley & Sons (1987-1997), or the like.

The PTGDR gene or the PTGER2 gene can be used in the form of a recombinant vector having such a gene incorporated therein. Examples of the vector to be used herein may include viral vectors and vectors for expression in animal cells. Preferably, viral vectors are used. Examples of such viral vector include retroviral vectors, adenoviral vectors, adeno-associated virus vectors, baculovirus vectors, vaccinia virus vectors, and lentivirus vectors. Of these, retroviral vectors are particularly preferred to use, since retroviral vectors enable stable and long-term expression of a foreign gene that had been incorporated into such vectors, through incorporation of the virus genome into a host chromosome after infection into cells.

Examples of the vector for expression in animal cells to be used herein may include pCXN2 (Gene, 108, 193-200, 1991), PAGE207 (JP Patent Publication (Kokai) No. 6-46841 (1994)) or variants thereof.

The above recombinant vector can be produced through transfection into an appropriate host to effect transformation, followed by culturing of thus obtained transformant. When the recombinant vector is a viral vector, animal cells capable of producing viruses are used as the host to be transfected with such a viral vector. For example, COS-7 cells, CHO cells, BALB/3T3 cells, and HeLa cells are use. Examples of the host to be used for retroviral vectors include ψCRE, ψCRIP, and MLV. Examples of the hosts to be used for adenoviral vectors or adeno-associated virus vectors include human embryonic kidney-derived 293 cells. Viral vectors can be transfected into animal cells by a calcium phosphate method. Moreover, when the recombinant vector is a vector for expression in animal cells, the E. coli K12 strain, the HB101 strain, and the DH5α strain, or the like can be used as the host to be transfected with such a vector. Transformation of E. coli is publicly known to those skilled in the art.

Thus obtained transformant is cultured in an appropriate medium under appropriate culture conditions, respectively. For example, a transformant of E. coli can be cultured using a liquid medium at a pH of about 5 to 8 containing carbon sources, nitrogen sources, inorganic substances, and the like which are essential for growth. The culture is normally carried out at 15° C. to 43° C. for about 8 to 24 hours. In this case, the recombinant vector of interest can be obtained through usual DNA isolation and purification methods, on completion of culture.

Moreover, transformants of animal cells can be cultured using a medium such as a 199 medium, an MEM medium, or a DMEM medium containing about 5% to 20% fetal bovine serum. The pH of the medium is preferably about 6 to 8. The culture is normally carried out at 30° C. to 40° C. for about 18 to 60 hours. In this case, since virus particles containing a target recombinant vector are released into a culture supernatant, the recombinant vector can be obtained through concentration and purification of the virus particles by a cesium chloride centrifugation method, a polyethylene glycol precipitation method, a concentration method using a filter, or the like.

The cell growth inhibitor of the present invention can be produced by mixing the abovementioned gene serving as an active ingredient with a base that is commonly used for gene therapeutic agents. Moreover, when such a gene is incorporated into a viral vector, virus particles containing the recombinant vector are prepared, and are then mixed with a base that is commonly used for gene therapeutic agents.

As to the base to be used for mixing the abovementioned gene or protein serving as an active ingredient, bases commonly used for injectable agents can be used. Examples thereof include: distilled water: salt solutions containing sodium chloride, a mixture of sodium chloride and mineral salts, or the like: solutions of mannitol, lactose, dextran, glucose, or the like: amino acid solutions of glycine, arginine, or the like: and mixed solutions having glucose solution with an organic acid or salt solution. Alternatively, these bases can also be prepared into injectable agents in the form of a solution, suspension, or dispersion, with use of auxiliary agents such as an osmoregulator, a pH adjuster, a vegetable oil, and a surfactant, in accordance with usual methods which are already known to those skilled in the art. These injectable agents can also be prepared in the form of a pharmaceutical preparation to be dissolved at the time of use, through operations such as powderization or lyophilization.

The form of administration of the cell growth inhibitor of the present invention may be either systemic administration such as usual intravenous administration and intraarterial administration, or-local administration such as local injection and oral administration. Furthermore, administration of the cell growth inhibitor may also take a combined form with catheterization, gene introduction technology, or surgical operation.

The administration dose of the cell growth inhibitor of the present invention varies depending on the age and gender of the patient, the symptom, the administration route, the frequency of administration, and the dosage form. Generally, the daily dose for an adult is within a range of about 1 μg/kg of body weight to 1000 mg/kg of body weight, and preferably a range of about 10 μg/kg of body weight to 100 mg/kg of body weight, in terms of weight of recombinant gene. The frequency of administration is not particularly limited.

### (3) Method for Activating Cell Growth and Cell Growth Activating Agent

According to the present invention, there are provided a method for activating cell growth which comprises introducing an siRNA, an shRNA, an antisense oligonucleotide, or a loss-of-function type gene of the PTGDR gene or the PTGER2 gene into tumor cells in vitro, and a cell growth activating agent which comprises said siRNA, shRNA, antisense oligonucleotide, or loss-of-function type gene.

siRNA is a double-strand RNA having a length of about 20 nucleotides (for example, 21 to 23 nucleotides) or shorter. Expression of such an siRNA in a cell enables to suppress the expression of a gene targeted by the siRNA (PTGDR gene or PTGER2 gene in the present invention).

The siRNA to be used in the present invention may take any form as long as it is capable of inducing RNAi. Here, the term “siRNA” is an abbreviation for “short interfering RNA”, which refers to a short-chain double-strand RNA of 10 nucleotides or longer obtained by: chemical or biochemical synthesis in an artificial manner; in vivo synthesis; or in vivo degradation of double-strand RNA of about 40 nucleotides or longer. The siRNA normally has a structure comprising 5′-phosphoric acid and 3′-OH, where the 3′ terminal projects by about 2 nucleotides. A specific protein binds to the siRNA to form RISC (RNA-induced-silencing-complex). This complex recognizes mRNA having the homologous sequence to that of siRNA and binds thereto. Then, the mRNA is cleaved at the central part of the siRNA with an RNase III-like enzymatic activity.

The siRNA sequence and the mRNA sequence being the target of cleavage preferably match 100%. However, such 100% match is not always required, when unmatched nucleotides are located away from the central part of the siRNA. This is because the RNAi cleaving activity often partially remains.

Preferably, the homologous region between the siRNA nucleotide sequence and the nucleotide sequence of the PTGDR gene or the PTGER2 gene whose expression has to be suppressed, does not include the translation initiation region of the concerned gene. Since various transcriptional factors and translational factors are predicted to bind to the translation initiation region, it is anticipated that the siRNA be unable to effectively bind to the mRNA, leading to lowered effect. Accordingly, the homologous sequence is preferably away from the translation initiation region of the concerned gene by 20 nucleotides, and more preferably by 70 nucleotides. The homologous sequence may be, for example, a sequence in the vicinity of the 3′ terminal of the concerned gene.

According to another aspect of the present invention, an shRNA (short hairpin RNA) comprising a short hairpin structure having a projection at the 3′ terminal may also be used as a factor which can suppress the expression of a target gene through RNAi. The term shRNA refers to a molecule of about 20 or more nucleotides, in which the single-strand RNA includes partially palindromic nucleotide sequences to thereby have a double-strand structure within the molecule, forming a hairpin-like structure. Such an shRNA is broken down into a length of about 20 nucleotides (typically 21 nucleotides, 22 nucleotides, and 23 nucleotides, for example) within a cell after being introduced into the cell, and thus is capable of inducing RNAi in a similar manner to that of siRNA. As described above, the shRNA induces RNAi in a similar manner to that of siRNA, and thus can be effectively used in the present invention.

The shRNA preferably has a projection at the 3′ terminal. There is no particular limitation on the length of the double-strand portion, although it is preferably about 10 or more nucleotides, and more preferably about 20 or more nucleotides. Here, the projecting 3′ terminal is preferably a DNA, more preferably a DNA of at least 2 or more nucleotides, and yet more preferably a DNA of 2 to 4 nucleotides.

As described above, in the present invention, siRNA or shRNA can be used as a factor which can suppress the expression of the PTGDR gene or the PTGER2 gene through RNAi. The advantages of siRNA are such that: (1) RNA itself, even when introduced into a cell, is not incorporated into a chromosome of normal cell, and therefore the treatment do not cause any inheritable mutations and the safety is high; (2) it is relatively easy to chemically synthesize short-chain double-strand RNA, and the form of double-strand RNA is more stable; and the like. The advantages of shRNA are such that: treatment through long-term suppression of gene expression can be achieved by producing a vector which can transcribe shRNA within a cell and introducing such a vector into the cell; and the like.

The siRNA or shRNA to be used in the present invention which can suppress the expression of the PTGDR gene or the PTGER2 gene through RNAi, may be chemically synthesized in an artificial manner, and may also be produced through in vitro RNA synthesis using DNA of a hairpin structure in which a sense strand DNA sequence and an antisense strand DNA sequence are linked in opposite directions, with a T7 RNA polymerase. In the case of in vitro synthesis, antisense and sense RNAs can be synthesized from a template DNA using the T7 RNA polymerase and a T7 promoter. After in vitro annealing thereof, transfection of the resultant RNA into cells induces RNAi to suppress the expression of a target gene. Here, for example, transfection of such RNA into cells can be carried out by a calcium phosphate method or a method using various transfection reagents (such as oligofectamine, lipofectamine, and lipofection).

The abovementioned siRNA and shRNA are also useful as cell growth activating agents. The administration method of the cell growth activating agent of the present invention may include oral administration, parenteral administration (such as intravenous administration, intramuscular administration, subcutaneous administration, intradermal administration, transmucosal administration, intrarectal administration, intravaginal administration, local administration to affected area, and skin administration), and direct administration to affected area. The agent of the present invention, if used as a medical composition, may be mixed with a pharmaceutically acceptable additive as required. Specific examples of such a pharmaceutically acceptable additive include, but not limited to, an antioxidant, a preservative, a coloring agent, a flavoring agent, a diluent, an emulsifier, a suspending agent, a solvent, a filler, an extending agent, a buffer agent, a delivery vehicle, a carrier, an excipient, and/or a pharmaceutical adjuvant.

The form of the pharmaceutical preparation of the agent of the present invention is not particularly limited, and examples thereof include a liquid agent, an injectable agent, and a sustained release agent. A solvent to be used for prescribing the agent of the present invention as the above pharmaceutical preparation may be either aqueous or non-aqueous.

Furthermore, the siRNA or shRNA serving as an active ingredient of the cell growth activating agent of the present invention can be administered in the form of a nonviral vector or a viral vector. In the case of a nonviral vector, there can be employed methods in which nucleic acid molecules are introduced using liposomes (such as a liposome method, an HVJ-liposome method, a cationic liposome method, a lipofection method, and a lipofectamine method), microinjection methods, methods in which nucleic acid molecules are transferred together with carriers (metal particles) into cells using a gene gun. If the siRNA or shRNA is administered in vivo using a viral vector, viral vectors such as a recombinant adenovirus and a recombinant retrovirus can be employed. Introduction of siRNA or shRNA gene into a cell or tissue can be achieved through introduction of DNA which expresses siRNA or shRNA into a detoxified DNA or RNA virus such as retrovirus, adenovirus, adeno-associated virus, herpes virus, vaccinia virus, poxvirus, poliovirus, Sindbis virus, Sendai virus, and SV40, followed by infection with the recombinant virus into the cell or tissue.

The dose of the cell growth activating agent of the present invention can be determined by those skilled in the art with a consideration of the purpose of administration, the disease severity, the age, weight, gender, and previous history of the patient, and the type of siRNA or shRNA serving as an active ingredient. The dose of siRNA or shRNA is not particularly limited, and examples thereof include about 0.1 ng/kg/day to about 100 mg/kg/day, and preferably about 1 ng/kg/day to about 10 mg/kg/day. RNAi effect is typically exerted for one to three days after the administration. Therefore, administration is preferably performed at a frequency of everyday to every third day. When an expression vector is used, the administration can be performed approximately once a week.

In the present invention, an antisense oligonucleotide can also be used as a cell growth activating agent. Antisense oligonucleotides to be used in the present invention are nucleotides that are complementary or hybridize to consecutive 5 to 100 nucleotide sequences within the DNA sequence of the PTGDR gene or the PTGER2 gene. Such an antisense oligonucleotide may be either DNA or RNA, or may also be modified as long as its functions remain unaffected. The term “antisense oligonucleotide” used in this description includes not only oligonucleotides wherein all nucleotides corresponding to nucleotides composing a predetermined DNA or mRNA region are complementary to their counterparts, but also oligonucleotides that contain some mismatching nucleotides, as long as such oligonucleotides can stably hybridize to DNA or mRNA.

In addition, the antisense oligonucleotides may be modified. After appropriate modification, resulting modified antisense oligonucleotides will be hardly degraded in vivo. This enables more stable inhibition of ITIIα. Examples of such modified oligonucleotide include S-oligo type (phosphorothioate-type), C-5 thyazole type, D-oligo type (phosphodiester-type), M-oligo type (methylphosphonate-type), peptide nucleic acid type, phosphodiester binding type, C-5 propinyl pyrimidine type, 2-O-propylribose, and 2′-methoxyribose type antisense oligonucleotides. Furthermore, such antisense oligonucleotide may also be an antisense oligonucleotide wherein at least some of the oxygen atoms composing phosphate groups are substituted with sulfur atoms or otherwise modified. Such an antisense oligonucleotide is particularly excellent in terms of nuclease resistance, water solubility, and affinity for RNA. As such an antisense oligonucleotide wherein at least some of the oxygen atoms composing phosphate groups are substituted with sulfur atoms or otherwise modified, an S-oligo type oligonucleotide can be enumerated.

The number of nucleotides in such antisense oligonucleotide is preferably 50 or less and more preferably 25 or less. Too large number of nucleotides results in increased effort and cost in oligonucleotide synthesis and lowered yields. Furthermore, the number of nucleotides of such antisense oligonucleotide is 5 or more and preferably 9 or more. A number of nucleotides of 4 or less is undesirable because of the resulting lowered specificity to a target gene.

Such antisense oligonucleotide (or a derivative thereof) can be synthesized by a usual method. For example, an antisense oligonucleotide or a derivative thereof can be easily synthesized using a commercially available DNA synthesizer (such as one produced by Applied Biosystems). It can be obtained by a synthesis method such as a solid-phase synthesis method using phosphoroamidite or a solid-phase synthesis method using hydrogen phosphonate.

When an antisense oligonucleotide is used as a cell growth activating agent in the present invention, it is generally provided in the form of a medical composition containing the antisense oligonucleotide and additive(s) for pharmaceutical preparation (such as a carrier and an excipient). The antisense oligonucleotide can be administered as a medicament to mammals including humans. The route of administration for such an antisense oligonucleotide is not particularly limited and may be either of oral administration or parenteral administration (such as intramuscular administration, intravenous administration, subcutaneous administration, peritoneal administration, transmucosal administration in the nasal cavity or the like, and inhalation administration).

The form of the pharmaceutical preparation of such an antisense oligonucleotide is not particularly limited. Examples of the pharmaceutical preparation for oral administration include tablets, capsules, fine granules, powders, granules, liquids, and syrups. Examples of the pharmaceutical preparation for parenteral administration include injections, infusions, suppositories, inhalants, transmucosal absorption systems, transdermal absorption systems, nasal drops, and ear drops. The form of a drug containing the antisense oligonucleotide, additive(s) to be used for the pharmaceutical preparation, a method for producing the pharmaceutical preparation, and the like can be appropriately selected by those skilled in the art.

The dose of the antisense oligonucleotide can be appropriately determined with a comprehensive consideration of the gender, age, and weight of the patient, the symptom severity, the purpose of administration such as prevention or treatment, and the presence/absence of other complication symptoms. The dose is generally 0.1 μg/kg of body weight/day to about 100 mg/kg of body weight/day, and preferably 0.1 μg/kg of body weight/day to about 10 mg/kg of body weight/day.

Furthermore, in the present invention, a loss-of-function type gene of the PTGDR gene or the PTGER2 gene can also be used as a cell growth activating agent. The loss-of-function type gene refers to a mutated gene which causes loss of function of the corresponding gene. Specific examples thereof include genes which translate proteins lacking their original functions, generally called muteins, including those lacking at least one constituent amino acid(s), those having at least one constituent amino acid(s) replaced by other amino acid(s), and those added with at least one amino acid(s), within the amino acid sequence produced by the concerned gene.

When such a loss-of-function type gene is used as the cell growth activating agent, it can be produced by mixing the abovementioned gene serving as an active ingredient with a base that is commonly used for gene therapeutic agents. Moreover, when such a gene is incorporated into a viral vector, virus particles containing the recombinant vector are prepared, and are then mixed with a base that is commonly used for gene therapeutic agents.

As to the base, bases commonly used for injectable agents can be used. Examples thereof include: distilled water: salt solutions containing sodium chloride, a mixture of sodium chloride and mineral salts, or the like: solutions of mannitol, lactose, dextran, glucose, or the like: amino acid solutions of glycine, arginine, or the like: and mixed solutions having glucose solution with an organic acid solution or salt solution. Alternatively, these bases can also be prepared into injectable agents in the form of a solution, suspension, or dispersion, with use of auxiliary agents such as an osmoregulator, a pH adjuster, a vegetable oil, and a surfactant, in accordance with usual methods which are already known to those skilled in the art. These injectable agents can also be prepared in the form of a pharmaceutical preparation to be dissolved at the time of use, through operations such as powderization or lyophilization.

The form of administration of the loss-of-function allele may be either systemic administration such as usual intravenous administration and intraarterial administration, or local administration such as local injection and oral administration. Furthermore, administration may also take a combined form with catheterization, gene introduction technology, or surgical operation.

The administration dose of the loss-of-function type gene varies depending on the age and gender of the patient, the symptom, the administration route, the frequency of administration, and the dosage form. Generally, the daily dose for an adult is within a range of about 1 μg/kg of body weight to 1000 mg/kg of body weight, and preferably a range of about 10 μg/kg of body weight to 100 mg/kg of body weight, in terms of weight of recombinant gene. The frequency of administration is not particularly limited.

Moreover, the abovementioned various gene therapeutic agents of the present invention can also be produced by adding a gene into a suspension of liposomes prepared by a usual method, followed by freezing and subsequent thawing. Examples of the method for preparing liposomes include a membrane shaking method, a sonication method, a reverse phase evaporation method, and a surfactant removal method. The suspension of liposomes is preferably subjected to sonication treatment before addition of a gene, so as to improve the efficiency of encapsulation of the gene. The liposomes having the gene encapsulated therein may be intravenously administered either directly or in the form of a suspension with water, physiological salt solution, or the like.

### (4) Screening Method of Antitumor Substance

As described above, it is considered that inactivation of the PTGDR gene or the PTGER2 gene serves as a main causative factor of neuroblastoma, and that drugs which normalize functions of such genes can be used as antitumor agents for neuroblastoma. In particular, if the causative factor of such inactivation is methylation of the CpG island of the PTGER2 gene and/or hypoacetylation of the histone H3 or H4 protein, drugs capable of relieving/relaxing these causative factors are useful as antitumor agents.

Therefore, as described above, the present invention provides a screening method of a drug having a demethylating function, namely present screening method 1; and a screening method of a drug having an acetylation enhancing function, namely present screening method 2.

As the premise for performing these present screening methods, neuroblastoma cells showing lowered expression level of the PTGER2 gene in specimen cells have to be obtained. In other words, the present screening method 1 requires a “neuroblastoma cell line showing lowered expression of the PTGER2 gene due to methylation of CpG island of the PTGER2 gene”, and the present screening method 2 requires a “neuroblastoma cell line showing lowered expression of the PTGER2 gene due to hypoacetylation of the histone H4 protein”. The method for establishing these cell lines can be carried out in accordance with usual methods, based on the abovementioned understandings. For example, such desirable “neuroblastoma cell line showing lowered expression of the PTGER2 gene due to methylation of CpG island of the PTGER2 gene (hereinunder, also referred to as methylated cancer cell line)” can be established by the following manner. Among at least cells exhibiting inactivation of the PTGER2 gene, cells showing restoration of the PTGER2 gene level by treatment with an already-known demethylating reagent (such as 5-azadeoxycytidine) are selected, and subjected to passage culture. Moreover, desirable “neuroblastoma cell line showing lowered expression of the PTGER2 gene due to hypoacetylation of the histone H3 or H4 protein (hereinunder, also referred to as deacetylated cancer cell line)” can be established by the following manner. Among at least neuroblastoma cells exhibiting methylation of the PTGER2 gene, cells showing restoration of the PTGER2 gene level by treatment with an already-known acetylation enhancer (such as Trichostatin A) are selected, and subjected to passage culture.

In the present screening method, test substances have to be contacted with the abovementioned methylated cancer cell line or deacetylated cell line. The form of such contact is not specifically limited. The contact can be achieved by adding a test substance, preferably diluted at an appropriate dilution strength, to the culture product of the methylated cell line, and subsequent culturing thereof. The PTGER2 gene expression level in the methylated cancer cell line or deacetylated cell line is quantified before and after addition of the test substance. Preferably, the quantification is performed with the passage of time. The difference in the gene expression level throughout the quantification is compared to that of a control culture product that has been cultured without addition of the test substance under the same condition. If the gene expression level of the culture product with addition of the test substance is higher than that of the control culture product, the test substance is selected as an “antitumor substance capable of activating the PTGER2 gene through demethylation of CpG island of the PTGER gene, in the reagent (1) using a methylated cancer cell line (present screening method 1); and the test substance is selected as an “antitumor substance capable of activating the PTGER2 gene through enhancement of acetylation of the histone H3 or H4 protein, in the test (2) using a deacetylated cancer cell line (present screening method 2).

Furthermore, preferably, the number of substances to be screened for as desirable antitumor ingredients for neuroblastoma by the present screening method is narrowed down to final candidates through additional in vivo screening, such as a screening method in which a growth inhibitory effect on neuroblastoma cells in nude mice transplanted with the abovementioned methylated cancer cell line or deacetylated cell line, and improvement in the viability of such nude mice, are used as indexes.

The present invention is hereafter described in greater detail with reference to the following examples, although the technical scope of the present invention is not limited thereto.

### EXAMPLE 1

### Inactivation of PTGER2 Gene in Neuroblastoma

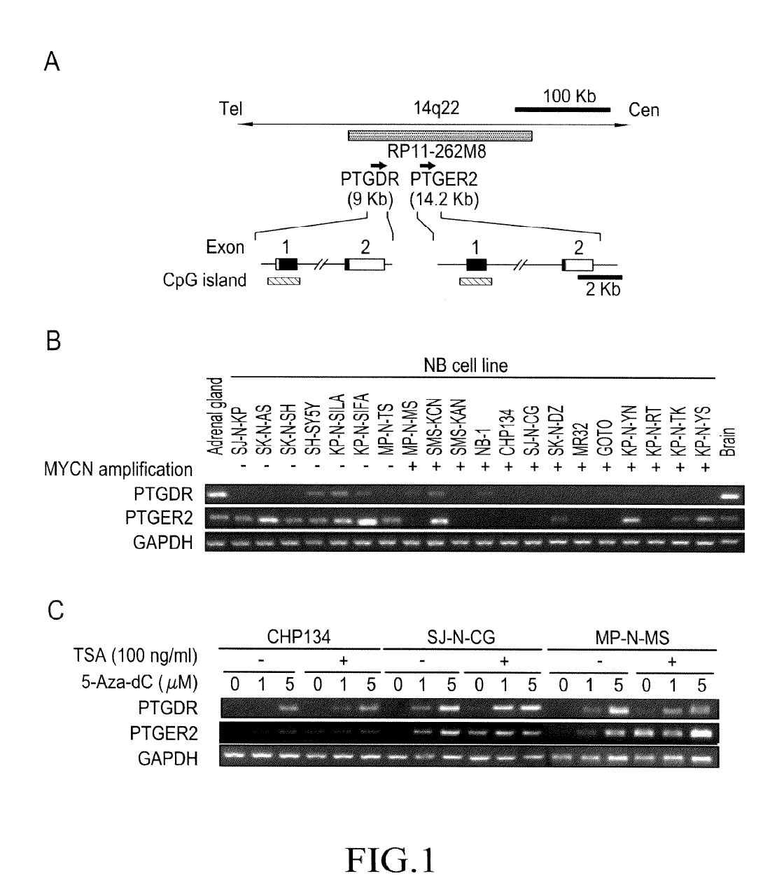

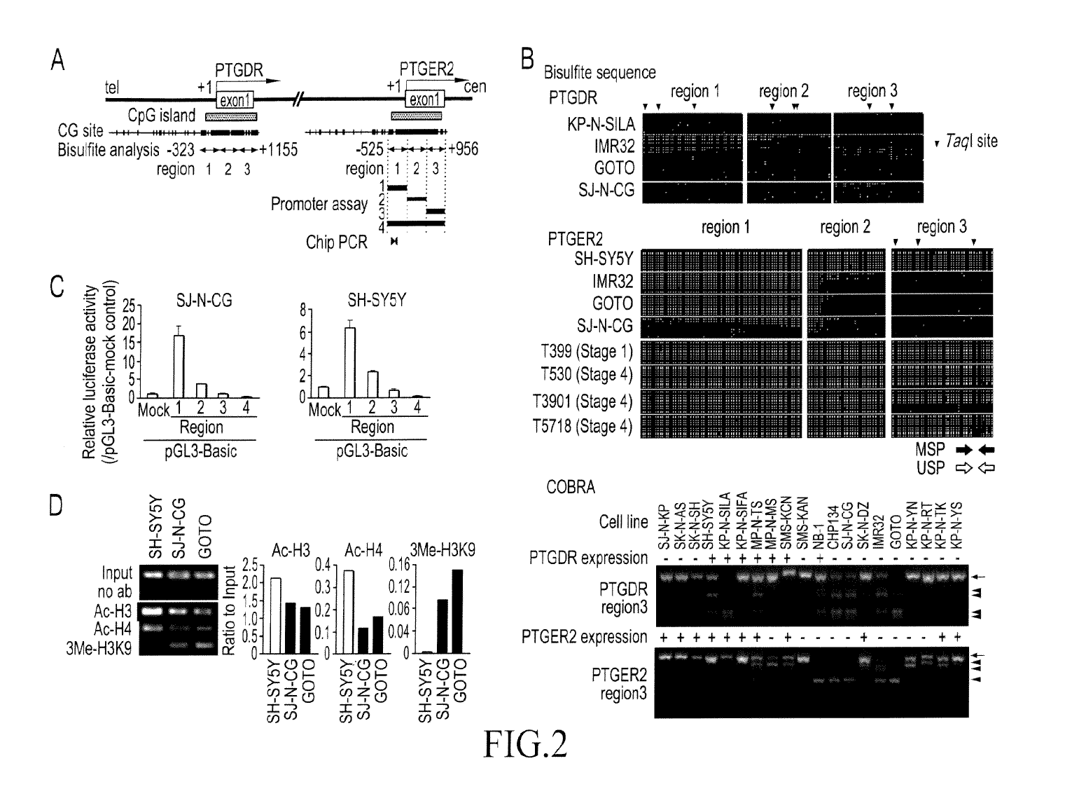

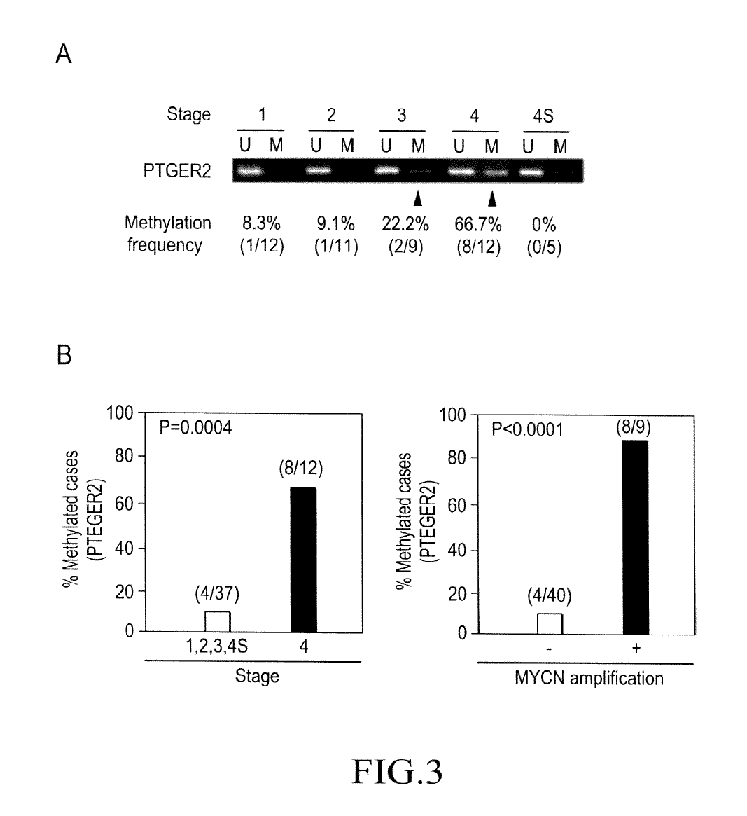

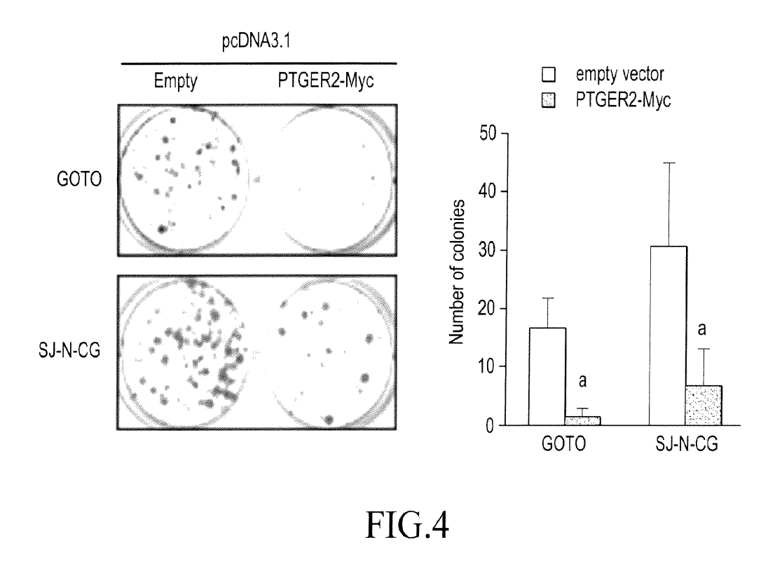

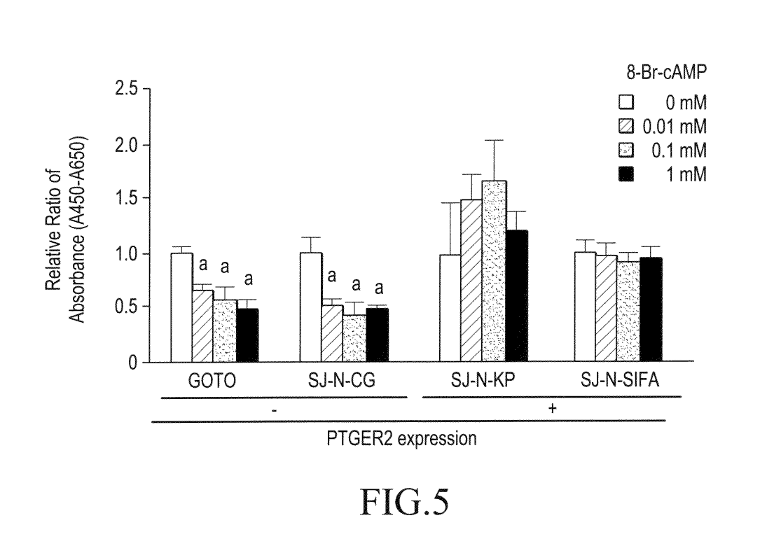

In order to detect novel gene alteration in neuroblastoma, genomic DNAs prepared from two types of neuroblastoma cell lines (IMR32 cells and GOTO cells) were subjected to BAMCA analysis using an MGC Whole Genome Array-4500 (Inazawa J., et al., Cancer Sci, 95, 559-563, 2004). As the control, genomic DNAs prepared from five types of Stage I neuroblastoma were used in mixture, which were labeled with Cy5. As the test DNA, genomic DNAs prepared from IMR32 cells and GOTO cells were used, which were labeled with Cy3. Specifically, the labeling was performed by binding an adaptor oligonucleotide to each genomic DNA (5 μg) that was digested with SmaI (100 units) and XmaI (20 units), followed by PCR reaction using primer sets complementary to the adaptor, Cy3-dCTP, and Cy5-dCTP (GE Healthcare). After the hybridization through BAMCA method, Cy3- and Cy5-derived fluorescence was monitored with the CGH-array using the GenePix 4000B scanner (Axon Instruments, CA, USA). The obtained results were analyzed using the GenePix Pro 4.1 imaging software (Axon Instruments, CA, USA). Averages of the Cy3- and Cy5-derived fluorescence intensities were adjusted to have the same values, and the Cy3/Cy5 ratio was obtained. When there is no genomic abnormality, the ratio is 1. Determination was performed such that a ratio of 1.5 or higher indicates the presence of genomic alteration. As a result, it was determined that genomic alteration was confirmed in one clone (RP11-262M8) existing in the chromosome 14q22.1 region. It was confirmed that the chromosomal region has two genes [PTGDR gene (NM000953) and PTGER2 gene (NM000956)] by the human genome database (http://genome.ucsc.edu/). The schematic diagram of the genomic structure is shown in FIG. 1A.

Expressions of these two genes were confirmed by RT-PCR using 20 types of neuroblastoma cell lines (FIG. 1B). As a result, all neuroblastoma cell lines which did not express the PTGER2 gene showed amplification of MYCN gene.

Moreover, in order to investigate whether or not the lowered expression of the PTGER2 gene was due to DNA methylation, neuroblastoma cell lines lacking expression of the PTGER2 gene were used and treated with 1 μM or 5 μM of 5-aza-dCyd, a demethylating reagent, for 5 days and/or 100 ng/ml of TSA, a deacetylation inhibitor, for 12 hours. RNA was extracted from each cell line and the PTGER2 gene expression thereof was examined by RT-PCR (FIG. 1C). As a result, it was found that the PTGER2 gene expression was restored by treatment with TSA alone, 5-aza-dCyd alone, and the combination thereof. The result apparently suggests that the lowered expression of the PTGER2 gene is associated with DNA methylation and histone acetylation.

### EXAMPLE 2

### Methylation Analysis of CpG Island of PTGER2 Gene

Methylation of a CpG island is one of the mechanisms which suppress gene expression. The CpG island of the PTGER2 gene was analyzed using CpGPLOT program (http://www.ebi.ac.uk/emboss/cpgplot/). As a result, it was confirmed the CpG island is present around exon 1 of the PTGER2 gene (FIG. 2A).

The exon 1 was divided into three regions, and the methylation status thereof was examined by bisulfite sequencing (Toyota M., et al., Cancer Res. 59, 2307, 1999) (FIG. 2B). For the methylation analysis, the EZ DNA methylation kit (Zymo RESEARCH, CA, USA) was used, in which neuroblastoma cell-derived genomic DNAs (2 μg) were treated with sodium bisulfite at 50° C. overnight and were subjected to PCR with primers designed to amplify methylated DNA of interest. The obtained PCR products were digested with TaqI restriction enzyme (New England BioLabs). The methylation status was monitored by utilizing the property of TaqI which does not digest sequences having unmethylated cytosines being modified with sodium bisulfite but does digest sequences having methylated cytosines being unmodified with sodium bisulfite. The PCR fragments were electrophoresed, and then the density ratio of bands of methylated fragments to bands of unmethylated fragments was measured with a densitometry using MultiGauge2.0 (Fujifilm Corporation). The methylation status of methylated regions was expressed as %. Moreover, these sequences were subcloned into a TOPO TA cloning vector (Invitrogen) to determine their nucleotide sequences. As a result, three neuroblastoma cell lines lacking expression of the PTGER2 gene (IMR32, GOTO, and SJ-N-CG) showed highly frequent methylation in the exon 1 region (Regions 2 and 3).

Moreover, in order to investigate the promoter activity of the CpG island of the PTGER2 gene that had been identified in FIG. 2A, the CpG island including the vicinity thereof was divided into three fragments. These fragments were inserted into a luciferase reporter plasmid (pGL3-Basic vector: Promega) and resulting plasmids were transfected into neuroblastoma cell lines (SJ-N-CG and SH-SY5Y). Through measurement of the luciferase activity using the Dual-Luciferase reporter assay system (Promega) in accordance with its instruction manual, the luciferase activity derived from the pGL3 vector having each Region was determined. As a result, it was found that the luciferase activity in Region 1 was high (FIG. 2C). Since the highly methylated Region 3 did not show a high luciferase activity, it may not be directly associated with inactivation of the gene expression.

The results suggest that epigenetic mechanisms other than DNA methylation might be associated with the regulation of expression of the PTGER2 gene. Therefore, histone acetylation and/or methylation were examined using ChIP assays (Sonoda, I., et al., Cancer Res, 64, 3741-3747, 2004). FIG. 2A shows the position of primers used for the ChIP assays, and FIG. 2D shows the results thereof. As a result, acetylated histone H3- and histone H4-binding DNA fragments were decreased in the PTGER2 gene-nonexpressing cell lines (SJ-N-CG and GOTO), as compared with the PTGER2 gene-expressing cell line (SH-SY5Y). However, DNA fragment which was bound to histone H3 where lysine 9 was trimethylated was increased in the SJ-N-CG and GOTO cell lines. These results suggest that, in neuroblastoma cells lacking expression of the PTGER2 gene, histone H3 and histone H4 are hypoacetylated, histone H3 lysine 9 is methylated, and further Region 3 of CpG island of the PTGER2 gene is methylated. It is suggested that these statuses regulate the expression of the PTGER2 gene.

### EXAMPLE 3

### Methylation of CpG Island of PTGER2 Gene in Primary Neuroblastoma

So far, methylation of CpG island of the PTGER2 gene was analyzed in neuroblastoma cells. Then, the occurrence of similar phenomenon in actual neuroblastoma tissues was investigated. For the tissue, one “stage 1” neuroblastoma tissue and three “stage 4” neuroblastoma tissues were used for analysis with bisulfite sequencing (FIG. 2B). As a result, two “stage 4” neuroblastoma tissues showed high level methylation within Region 3, whereas the “stage 1” neuroblastoma tissue did not show any high level methylation.

Further, methylation of Region 3 was examined in 49 cases of neuroblastoma tissues through methylation-specific PCR (MSP) and subsequent examination of the PCR products with 3% agarose gel electrophoresis. As a result, methylation of Region 3 was detected in 12 of 49 cases (24.5%). Of the 12 cases, four cases were in stage 1, 2, 3 and 4S neuroblastoma, whereas other eight cases were in stage 4 neuroblastoma (FIG. 3A). Moreover, eight of nine cases (88.9%) with MYCN gene amplification showed methylation of Region 3 of the PTGER2 gene (FIG. 3B), whereas only four of 40 cases (10%) without MYCN gene amplification showed methylation of Region 3 of the PTGER2 gene. These results suggest that the detection of methylation of the PTGER2 gene and amplification of the MYCN gene enable ascertainment of the malignancy of neuroblastoma.

### EXAMPLE 4

### Suppression of Neuroblastoma Proliferation by Activation of PTGER2 Gene

Based on the aforementioned results, it was investigated whether activation of the PTGER2 gene expression would suppress proliferation of neuroblastoma. First, a plasmid expressing Myc-tagged PTGER2 gene (pcDNAPTGER2-Myc) was constructed. This can be used for monitoring the role of full-length PTGER2 gene. The plasmid was prepared by inserting the RT-PCR amplification product of PTGER2 cDNA into the pcDNA3.1 expression vector so as to have the translation frame along with the Myc tag. An empty vector (pcDNA3.1-mock) without PTGER2 gene was used as a control. These expression plasmids were mixed with a transfection regent, FuGENE6 (Roche Diagnostics), and were transfected into SJN-CG and GOTO cells. After two or three weeks, cells which grew under the presence of G418, a neomycin-like drug, were fixed with 70% ethanol and stained with crystal violet. Then, the number of colonies was counted. As a result, the number of colonies produced by pcDNA-PTGER2-Myc-transfected cells decreased markedly compared to empty vector-transfected cells (FIG. 4). The result apparently suggests that activation of the PTGER2 gene expression enables suppression of proliferation of neuroblastoma, and functions as a cancer suppressing agent.

### EXAMPLE 5

### Suppression of Neuroblastoma Proliferation by Increase of Intracellular cAMP Concentration

Since the PTGER2 protein and G protein are coupled to be associated with increased intracellular cAMP concentration (Narumiya, et al., Physiol Rev, 79, 1193-1226, 1999), the association between intracellular cAMP concentration and proliferation of neuroblastoma was investigated. First, neuroblastoma cell lines lacking expression of the PTGER2 gene (GOTO and SJ-N-CG) and neuroblastoma cell lines expressing the PTGER2 gene (SJ-N-KP and KP-N-SIFA) were cultured in a growth medium with 0 mM, 0.01 mM, 0.1 mM, and 1 mM of 8-Br-cAMP for 72 hours. Then, the viability of these cells was detected by WST assay (FIG. 5). As a result, it was revealed that when a PTGER2 gene-nonexpressing cell line was added with 8-Br-cAMP to increase its intracellular cAMP concentration, the proliferation of neuroblastoma lacking expression of the PTGER2 gene was suppressed. This suggests that accumulation of intracellular cAMP in neuroblastoma lacking expression of the PTGER2 gene functions as a cancer suppressing agent.

### EFFECT OF THE INVENTION

According to the present invention, it becomes possible to precisely understand signs of canceration and malignancy in neural-crest-derived cell specimen. Furthermore, proliferation of neuroblastoma can be suppressed by introducing a transcript of the PTGER2 gene that inactivates the gene expression in neuroblastoma. Furthermore, a therapeutic agent for neuroblastoma that is developed by inactivation of PTGER2 gene expression can be screened.

1. A method for detecting cancer, which comprises detecting canceration including malignancy of a specimen through detection of inactivation of a gene in the q22 region of chromosome 14 in the specimen.

2. The method for detecting cancer according to claim 1, wherein the gene is a PTGDR gene or a PTGER2 gene.

3. The method for detecting cancer according to claim 1, which further comprises detecting amplification of an MYCN gene in the specimen.

4. The method for detecting cancer according to claim 1, wherein the gene inactivation is inactivation due to methylation of a CpG island.

5. The method for detecting cancer according to claim 1, wherein the gene inactivation is inactivation due to acetylation status of a histone H4 protein or a histone H3 protein, or trimethylation status of histone H3K9.

6. The method for detecting cancer according to claim 1, wherein the specimen is a cell derived from the neural crest.

7. The method for detecting cancer according to claim 1, wherein cancer is neuroblastoma.

8. The method for detecting cancer according to claim 1, wherein the gene inactivation is detected by using a DNA chip method, a Southern blot method, a Northern blot method, a real-time RT-PCR method, a FISH method, a CGH method, an array CGH method, a bisulfite sequence method, or a COBRA method.

9. A method for inhibiting cell growth, which comprises introducing a PTGDR gene, a PTGER2 gene or a protein which is an expression product of said gene into cells in vitro.

10. A cell growth inhibitor, which comprises a PTGDR gene, a PTGER2 gene, or a protein which is an expression product of said gene.

11. A method for activating cell growth, which comprises introducing an siRNA, an shRNA, an antisense oligonucleotide, or a loss-of-function type gene of a PTGDR gene or a PTGER2 gene into tumor cells in vitro.

12. A cell growth activating agent, which comprises an siRNA, an shRNA, an antisense oligonucleotide, or a loss-of-function type gene of a PTGDR gene or a PTGER2 gene.

13. A method for inhibiting cell growth, which comprises in vitro accumulation of cAMP in a specimen.

14. A screening method of a substance, which comprises: contacting a test substance with a neuroblastoma cell showing lowered expression of a PTGDR gene or a PTGER2 gene due to methylation of a CpG island of the PTGDR gene or the PTGER2 gene; detecting expression of the PTGDR gene or the PTGER2 gene; and, if the gene expression thereof is higher than that of a system without the contact of the test substance, selecting the test substance as an antitumor substance capable of activating the PTGDR gene or the PTGER2 gene through demethylation of the CpG island of the PTGDR gene or the PTGER2 gene.

15. A screening method of a substance, which comprises: contacting a test substance with a neuroblastoma cell showing lowered expression of a PTGDR gene or a PTGER2 gene due to hypoacetylation of a histone H3 or H4 protein or methylation of a lysine residue at position 6 (K9) of a histone H3 protein; detecting expression of the PTGDR gene or the PTGER2 gene; and, if the gene expression thereof is higher than that of a system without the contact of the test substance, selecting the test substance as an antitumor substance capable of activating the PTGDR gene or the PTGER2 gene through enhancement of acetylation of the histone H4 protein.
